(19) United States
(12) Patent Application Publication
(10) Pub. No.: US 2021/0330572 A1
(43) Pub. Date: Oct. 28, 2021

(54) COSMETIC COMPOSITION COMPRISING AT LEAST ONE HYDROPHOBIC FILM-FORMING POLYMER AND AT LEAST ONE AMINOSILICONE

(71) Applicant: L'OREAL, Paris (FR)

(72) Inventors: Audrey CORREIA, Saint Ouen (FR); Cécile TOULOUZAN, Saint Ouen (FR)

(21) Appl. No.: 17/312,585

(22) PCT Filed: Dec. 20, 2019
(86) PCT No.: PCT/EP2019/086803

§ 371 Date: Jun. 10, 2021

(30) Foreign Application Priority Data
Dec. 20, 2018 (FR) 1873703

(51) Int. Cl. A61K 8/898; A61K 8/81; A61Q 5/12

(52) U.S. Cl. CPC A61K 8/898; A61K 8/8111; A61K 8/8117; A61K 2800/594; A61K 2800/5426; A61K 2800/5424; A61Q 5/12

(57) ABSTRACT

The present invention relates to a cosmetic composition comprising a combination of at least one hydrophobic film-forming polymer chosen from hydrocarbon-based resins, and of at least one aminosilicone in an organic solvent. The present invention also relates to a cosmetic process for treating keratin fibres, in particular human keratin fibres such as the hair, using said cosmetic composition. Finally, the present invention relates to the use of said cosmetic composition for conditioning keratin fibres, in particular human keratin fibres such as the hair.

The present invention relates to a cosmetic composition comprising a combination of at least one hydrophobic film-forming polymer and of at least one aminosilicone in an organic solvent.

The present invention also relates to a cosmetic process for treating keratin fibres, in particular human keratin fibres such as the hair, using said cosmetic composition.

Finally, the present invention relates to the use of said cosmetic composition for conditioning keratin fibres, in particular human keratin fibres such as the hair.

Hair is generally damaged and embrittled by the action of external atmospheric agents such as light and bad weather, and by mechanical or chemical treatments, such as brushing, combing, dyeing, bleaching, permanent-waving and/or relaxing.

Thus, in order to remedy these drawbacks, it is now common practice to pursue haircare treatments involving the use of care compositions that make it possible to condition the hair after these treatments in order notably to give it sheen, softness, suppleness, lightness, a natural feel and disentangling properties.

These haircare compositions may advantageously be compositions to be applied after shampooing and may be in the form of gels, hair lotions or more or less thick creams.

To improve the cosmetic properties of these compositions, it is known practice to introduce therein cosmetic agents, known as conditioning agents, intended mainly to repair or to limit the harmful or undesirable effects brought about by the various treatments or attacking factors to which hair fibres are more or less repeatedly subjected. These conditioning agents may, of course, also improve the cosmetic behaviour of natural hair.

With this aim, it has already been proposed to use cosmetically active organic compounds, such as cationic polymers and silicones, as conditioning agents in cosmetic care compositions, such as hair conditioners, in order to give the hair satisfactory cosmetic properties, in particular softness, suppleness, lightness, a natural feel and an improved ability to be disentangled.

However, the use of these compounds in care and/or conditioning cosmetic compositions does not afford the hair entirely satisfactory and lasting cosmetic properties. This is because these compositions generally provide cosmetic properties, such as the disentangling of wet and dry hair, suppleness, smoothness, sheen, coating and an individualized nature of the hair strands, which remain insufficient and which have a tendency to fade out after washing the hair with a standard shampoo.

Conventional haircare compositions also have a tendency to weigh down the hair and to cover it with a brittle, squeaky or crackly film. After application, these compositions may leave a greasy and coarse feel, which consumers find unpleasant.

In addition, these care compositions are not entirely satisfactory in terms of providing sheen to the head of hair.

Moreover, these care compositions change substantially over time under normal storage conditions. This is reflected by an unsatisfactory texture of the compositions.

Thus, there is a real need to provide a cosmetic composition for treating keratin fibres, in particular human keratin fibres such as the hair, which does not have the drawbacks mentioned above, i.e. which is notably capable of affording good conditioning properties, notably in terms of sheen, while at the same time giving the keratin fibres a sensation of lightness and softness (or a glidant appearance). These properties must not only be satisfactory from the very first application, but also lasting between two shampoo washes.

The composition must also remain stable over time.

It has been discovered, surprisingly, that a composition comprising a particular combination of at least one hydrophobic film-forming polymer and of at least one aminosilicone in at least one organic solvent makes it possible to achieve the objectives presented above.

One subject of the present application is thus a cosmetic composition comprising:

a) one or more hydrophobic film-forming polymers chosen from indene hydrocarbon-based resins, aliphatic pentanediene resins, mixed resins of pentanediene and of indene, diene resins of cyclopentanediene dimers, diene resins of isoprene dimers and mixtures thereof,

b) one or more aminosilicones, and

c) one or more organic solvents.

The combination of a hydrophobic film-forming polymer with an aminosilicone gives keratin fibres good conditioning properties, and notably suppleness, smoothness and sheen, while at the same time giving them a very good, non-greasy and non-laden, natural feel with a glidant coating. Thus, fibres treated with the composition according to the invention are not made heavy and have a light, pleasant sensation.

The composition according to the present invention also makes it possible to restore the natural sheen of damaged or degraded keratin fibres, or even to improve the sheen of keratin fibres that are not damaged or degraded.

In addition, the composition according to the invention can afford these conditioning properties on wet and dry hair, in rinse-out or leave-in application, with or without supplying heat brought notably by means of a hairdryer and/or a straightening iron.

Moreover, the conditioning properties afforded by the composition according to the invention show good resistance to the various attacking factors to which hair may be subjected, such as light, bad weather, washing and perspiration.

A subject of the present invention is also a cosmetic process for treating keratin fibres, in particular human keratin fibres such as the hair, comprising the application to said keratin fibres of an anhydrous cosmetic composition as defined previously.

The present invention also relates to the use of said anhydrous cosmetic composition for conditioning keratin fibres, in particular human keratin fibres such as the hair.

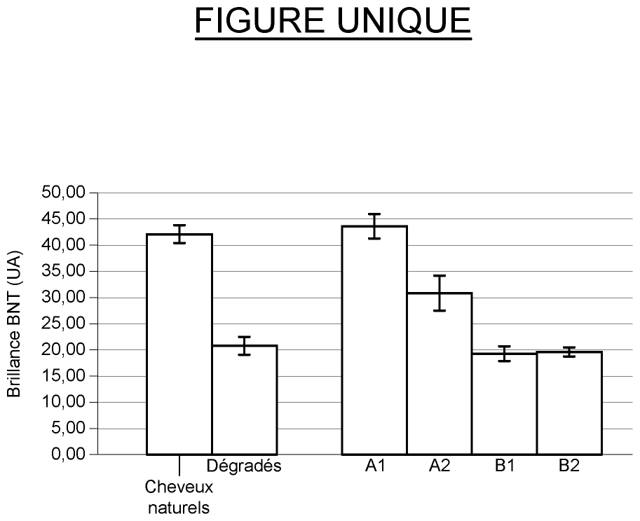

FIG. 1 shows the gloss measurements (expressed in arbitrary units) obtained for locks of hair treated with compositions according to the invention (A1 and A2) and compares them with those obtained for locks of hair treated with comparative compositions (B1 and B2). The gloss measurements on locks of natural and “degraded” chestnut-brown hair are also shown in this FIGURE.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

Hydrophobic Film Forming Polymers

The composition according to the present invention comprises (a) one or more hydrophobic film-forming polymers chosen from indene hydrocarbon-based resins, aliphatic pentanediene resins, mixed resins of pentanediene and of indene, diene resins of cyclopentanediene dimers, diene resins of isoprene dimers and mixtures thereof.

In the present patent application, the term “film-forming polymer” means a polymer that is capable, by itself or in the presence of an auxiliary film-forming agent, of forming a macroscopically continuous deposit, and preferably a cohesive deposit, and even better still a deposit whose cohesion and mechanical properties are such that said deposit can be isolated and manipulated individually, for example when said deposit is prepared by pouring onto a non-stick surface such as a Teflon-coated or silicone-coated surface.

The term “hydrophobic” or “water-insoluble” polymer means that the polymer is not soluble in water, according to the definition below.

The term “water-soluble” polymer means that the polymer dissolves in water or in a 50/50 by volume mixture of water and ethanol, or alternatively in a mixture of water and isopropanol, without modification of the pH, at a solids content of 5% by weight, at room temperature (25° C., 1 atm.). The polymer is considered to be soluble if it does not form a precipitate or agglomerate that is visible to the naked eye when it is placed in solution, and if it therefore gives a clear solution.

The composition comprises one or more hydrocarbon-based resins chosen from indene hydrocarbon-based resins, aliphatic pentanediene resins, mixed resins of pentanediene and of indene, diene resins of cyclopentanediene dimers, diene resins of isoprene dimers and mixtures thereof, as hydrophobic-forming polymers.

For the purposes of the invention, the term “hydrocarbon-based resin” refers to a resin formed solely from carbon and hydrogen atoms.

The hydrocarbon-based resin(s) are advantageously chosen from low molecular weight polymers that may be classified, according to the type of monomer they comprise, as:
indene hydrocarbon-based resins, preferably such as resins derived from the polymerization in major proportion of indene monomer and in minor proportion of a monomer chosen from styrene, methylindene and methylstyrene, and mixtures thereof. These resins may optionally be hydrogenated. These resins may have a molecular weight ranging from 290 to 1150 g/mol;

Examples of indene resins that may be mentioned include those sold under the reference Escorez 7105 by the company Exxon Chem., Nevchem 100 and Nevex 100 by the company Neville Chem., Norsolene 5105 by the company Sartomer, Picco 6100 by the company Hercules and Resinall by the company Resinall Corp., or the hydrogenated indene/methylstyrene/styrene copolymers sold under the name Regalite by the company Eastman Chemical, in particular Regalite R 1100, Regalite R 1090, Regalite R-7100, Regalite R1010 Hydrocarbon Resin and Regalite R1125 Hydrocarbon Resin;
aliphatic pentanediene resins such as those derived from the majority polymerization of the 1,3-pentanediene (trans- or cis-piperylene) monomer and of minor monomer(s) chosen from isoprene, butene, 2-methyl-2-butene, pentene and 1,4-pentanediene, and mixtures thereof. These resins may have a molecular weight ranging from 1000 to 2500 g/mol;

Such 1,3-pentanediene resins are sold, for example, under the references Piccotac 95 by the company Eastman Chemical, Escorez 1304 by the company Exxon Chemicals, Nevtac 100 by the company Neville Chem. or Wingtack 95 by the company Goodyear;
mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;
diene resins of cyclopentanediene dimers such as those derived from the polymerization of a first monomer chosen from indene and styrene, and of a second monomer chosen from cyclopentanediene dimers such as dicyclopentanediene, methyldicyclopentanediene and other pentanediene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;
diene resins of isoprene dimers such as terpenic resins derived from the polymerization of at least one monomer chosen from α-pinene, β-pinene and limonene, and mixtures thereof. These resins may have a molecular weight ranging from 300 to 2000 g/mol. Such resins are sold, for example, under the names Piccolyte A115 and S125 by the company Hercules or Zonarez 7100 or Zonatac 105 Lite by the company Arizona Chem.

According to a preferred embodiment, the hydrocarbon-based resin is chosen from hydrocarbon-based resins that are solid at room temperature (20° C.).

Preferably, the composition comprises at least one hydrocarbon-based resin such as those described previously, and notably chosen from indene hydrocarbon-based resins and aliphatic pentanediene resins, and mixtures thereof. According to a preferred embodiment, the hydrocarbon-based resin is chosen from indene hydrocarbon-based resins.

According to a preferred embodiment, the resin is chosen from hydrogenated indene/methyl styrene/styrene copolymers.

In particular, use may be made of hydrogenated indene/methylstyrene/styrene copolymers, such as those sold under the name Regalite by the company Eastman Chemical, such as Regalite R 1100 CG Hydrocarbon Resin, Regalite R 1100, Regalite R 1090, Regalite R-7100, Regalite R1010 Hydrocarbon Resin and Regalite R1125 Hydrocarbon Resin.

Preferably, the hydrocarbon-based resin(s) (a) are chosen from indene hydrocarbon-based resins, aliphatic pentanediene resins, and mixtures thereof, and better still from indene hydrocarbon-based resins.

The total content of said hydrophobic film-forming polymer(s) (a), present in the composition according to the invention preferably ranges from 0.1% to 20% by weight, preferentially from 1% to 15% by weight, more preferentially from 2% to 10% by weight and better still from 3% to 8% by weight, relative to the total weight of the composition.

Another object of the present invention relates to a cosmetic composition comprising:

a′) one or more hydrophobic film-forming polymers chosen from hydrocarbon-based block copolymers,

b) one or more aminosilicones, and

c) one or more organic solvents.

The term “block” polymer means a polymer comprising at least two different blocks and preferably at least three different blocks.

In this embodiment, the composition according to the invention comprises at least one hydrocarbon-based block copolymer, preferably a block copolymer that is soluble or dispersible in a liquid fatty phase.

The hydrocarbon-based block copolymer may notably be a diblock, triblock, multiblock, radial or star copolymer, or mixtures thereof.

For the purposes of the invention, the term “hydrocarbon-based polymer” refers to a polymer formed solely from carbon and hydrogen atoms.

Such hydrocarbon-based block copolymers are described in patent application US-A-2002/005 562 and in patent U.S. Pat. No. 5,221,534.

The copolymer may contain at least one block whose glass transition temperature is preferably less than 20° C., preferably less than or equal to 0° C., preferably less than or equal to −20° C. and more preferably less than or equal to −40° C. The glass transition temperature of said block may be between −150° C. and 20° C. and notably between 100° C. and 0° C.

The hydrocarbon-based block copolymer present in the composition according to the invention is a copolymer formed by polymerization of an olefin. The olefin may notably be an ethylenically unsaturated monomer.

Examples of olefins that may be mentioned include ethylenic carbide monomers, notably containing one or two ethylenic unsaturations and containing from 2 to 5 carbon atoms, such as ethylene, propylene, butadiene, isoprene or pentadiene. Advantageously, the hydrocarbon-based block copolymer is a block copolymer of styrene and olefin.

Block copolymers comprising at least one styrene block and at least one block comprising units chosen from butadiene, ethylene, propylene, butylene and isoprene or a mixture thereof are notably preferred.

According to a preferred embodiment, the hydrocarbon-based block copolymer is hydrogenated to reduce the residual ethylenic unsaturations after the polymerization of the monomers.

In particular, the hydrocarbon-based block copolymer is an optionally hydrogenated copolymer, containing styrene blocks and ethylene/C₃-C₄ alkylene blocks.

Diblock copolymers, which are preferably hydrogenated, that may be mentioned include styrene-ethylene/propylene copolymers, styrene-ethyl ene/butadi ene copolymers and styrene-ethylene/butylene copolymers. Diblock polymers are notably sold under the name Kraton® G1701E by the company Kraton Polymers. Triblock copolymers, which are preferably hydrogenated, that may be mentioned include styrene-ethylene/propylene-styrene copolymers, styrene-ethylene/butadiene-styrene copolymers, styrene-ethylene/butylene-styrene copolymers, styrene-isoprene-styrene copolymers and styrene-butadiene-styrene copolymers. Triblock polymers are notably sold under the names Kraton® G1650, Kraton® G1652, Kraton® D1101, Kraton® D1102 and Kraton® D1160 by the company Kraton Polymers.

According to one embodiment of the present invention, the hydrocarbon-based block copolymer is a styrene-ethylene/propylene diblock copolymer, notably such as the diblock polymers sold under the name Kraton® G1701E by the company Kraton Polymers.

When the composition comprises one or more hydrocarbon-based block copolymers (a′) as described above, the total content of said hydrocarbon-based block copolymer(s) (a′), present in the composition according to the invention, preferably ranges from 0.1% to 20% by weight, preferentially from 1% to 15% by weight, more preferentially from 2% to 10% by weight and better still from 3% to 8% by weight, relative to the total weight of the composition.

Aminosilicones

The composition according to the present invention also comprises (b) one or more aminosilicones.

The term “aminosilicone” denotes any silicone including at least one primary, secondary or tertiary amine or a quaternary ammonium group.

The weight-average molecular masses of these aminosilicones may be measured by gel permeation chromatography (GPC) at room temperature (25° C.), as polystyrene equivalent. The columns used are μ styragel columns. The eluent is THF and the flow rate is 1 ml/min. 200 μl of a 0.5% by weight solution of silicone in THF are injected. Detection is performed by refractometry and UV-metry.

Throughout the text hereinbelow, the term “silicone” is intended to denote, in accordance with what is generally accepted, any organosilicon polymer or oligomer of linear or cyclic, branched or crosslinked structure, of variable molecular weight, obtained by polymerization and/or polycondensation of suitably functionalized silanes, and formed essentially from a repetition of main units in which the silicon atoms are linked together via oxygen atoms (siloxane bond —Si—O—Si—), optionally substituted hydrocarbon-based groups being directly linked via a carbon atom to said silicon atoms. The hydrocarbon-based groups that are the most common are alkyl groups, notably C₁-C₁₀ alkyl groups and in particular methyl, fluoroalkyl groups, the alkyl part of which is C₁-C₁₀, and aryl groups and in particular phenyl.

The appropriate aminosilicones that may be used in accordance with the present invention comprise, without being limited thereto, volatile and non-volatile, cyclic, linear and branched aminosilicones, with a viscosity ranging from 5×10⁻⁶ to 2.5 m²/s at 25° C., for example from 1×10⁻⁵ to 1 m²/s.

Preferably, the aminosilicone(s) are chosen from:

a) the polysiloxanes corresponding to formula (I):

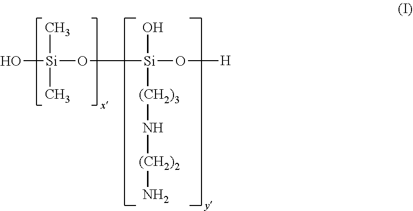

in which x′ and y′ are integers such that the weight-average molecular mass (Mw) is between 5000 and 500 000 g/mol;

b) the aminosilicones corresponding to formula (II):

R′ₐG₃₋ₐ-Si(OSiG₂)ₙ-(OSiG_{b}R′_{2-b})ₘ—O-SiG_{3-a′}-R′_{a′} (II)

in which:
G, which may be identical or different, denotes a hydrogen atom or a phenyl, OH, C₁-C₈ alkyl, for example methyl, or C₁-C₈ alkoxy, for example methoxy, group,
a and a′, which may be identical or different, denote 0 or an integer from 1 to 3, in particular 0, with the proviso that at least one from among a and a′ is equal to zero,
b denotes 0 or 1, in particular 1,
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
R′, which may be identical or different, denotes a monovalent radical of formula —C_{q}H_{2q}L in which q is a number ranging from 2 to 8 and L is an optionally quaternized amino group chosen from the following groups:
—NR″-Q-N(R″)₂,
—N(R″)₂,
—N⁺(R″)₃ A⁻,
—N⁺H(R″)₂ A⁻,
—N⁺H₂(R″) A⁻,
—NRS′-Q-N⁺(R″)H₂ A⁻,
—NR″-Q-N⁺(R″)₂H A⁻ and
—NRS′-Q-N⁺(R″)₃ A⁻,

in which R″, which may be identical or different, denotes hydrogen, phenyl, benzyl, or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A⁻ represents a cosmetically acceptable anion, notably a halide such as fluoride, chloride, bromide or iodide.

According to a first embodiment, the aminosilicones corresponding to formula (II) are chosen from the silicones known as “trimethylsilyl amodimethicone”, corresponding to formula (III):

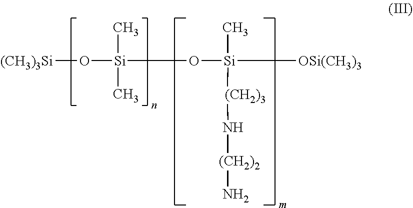

in which m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10.

According to a second embodiment, the aminosilicones corresponding to formula (II) are chosen from the silicones of formula (IV) below:

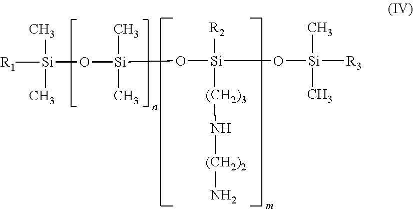

in which:
m and n are numbers such that the sum (n+m) ranges from 1 to 1000, notably from 50 to 250 and more particularly from 100 to 200; n denoting a number from 0 to 999, notably from 49 to 249 and more particularly from 125 to 175, and m denoting a number from 1 to 1000, notably from 1 to 10 and more particularly from 1 to 5; and
R₁, R₂ and R₃, which may be identical or different, represent a hydroxyl or C₁-C₄ alkoxy radical, at least one of the radicals R₁ to R₃ denoting an alkoxy radical. Preferably, the alkoxy radical is a methoxy radical.

The hydroxy/alkoxy mole ratio preferably ranges from 0.2:1 to 0.4:1, preferably from 0.25:1 to 0.35:1 and is more particularly equal to 0.3:1.

The weight-average molecular mass (Mw) of these silicones preferably ranges from 2000 to 1 000 000 g/mol and more particularly from 3500 to 200 000 g/mol.

According to a third embodiment, the aminosilicones corresponding to formula (II) are chosen from the silicones of formula (V) below:

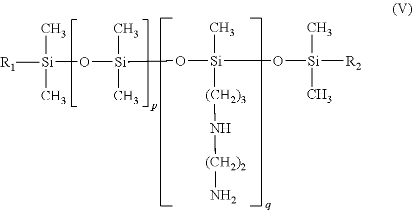

in which:
p and q are numbers such that the sum (p+q) ranges from 1 to 1000, in particular from 50 to 350 and more particularly from 150 to 250; p denoting a number from 0 to 999, notably from 49 to 349 and more particularly from 159 to 239, and q denoting a number from 1 to 1000, notably from 1 to 10 and more particularly from 1 to 5; and
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxy/alkoxy mole ratio generally ranges from 1:0.8 to 1:1.1, preferably from 1:0.9 to 1:1 and more particularly is equal to 1:0.95.

The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) may include in their composition one or more other aminosilicones, the structure of which is different from formula (IV) or (V).

A product containing aminosilicones of structure (IV) is sold by the company Wacker under the name Belsil® ADM 652.

A product containing aminosilicones of structure (V) is sold by the company Wacker under the name Fluid WR 1300®. Another product containing aminosilicones of structure (IV) is sold by Wacker under the name Belsil ADM LOG 1®.

When these aminosilicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-mean size of the silicone particles in the emulsion generally ranges from 3 nm to 500 nanometres. Preferably, notably as aminosilicones of formula (V), use is made of microemulsions of which the mean particle size ranges from 5 nm to 60 nm (limits included) and more particularly from 10 nm to 50 nm (limits included). Thus, use may be made according to the invention of the aminosilicone microemulsions of formula (V) sold under the names Finish CT 96 E® or SLM 28020® by the company Wacker.

According to a fourth embodiment, the aminosilicones corresponding to formula (II) are chosen from the silicones of formula (VI) below:

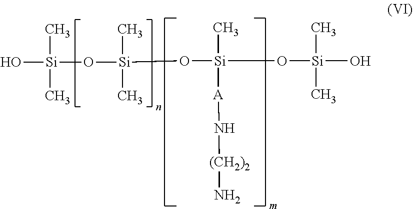

in which:
m and n are numbers such that the sum (n+m) ranges from 1 to 2000 and in particular from 50 to 150, n denoting a number from 0 to 1999 and notably from 49 to 149, and m denoting a number from 1 to 2000 and notably from 1 to 10; and
A denotes a linear or branched alkylene radical containing from 4 to 8 carbon atoms and preferably 4 carbon atoms. This radical is preferably linear.

The weight-average molecular mass (Mw) of these aminosilicones preferably ranges from 2000 to 1 000 000 g/mol and more particularly from 3500 to 200 000 g/mol.

A silicone corresponding to this formula is, for example, the Xiameter MEM 8299 Emulsion from Dow Corning.

According to a fifth embodiment, the aminosilicones corresponding to formula (II) are chosen from the silicones of formula (VII) below:

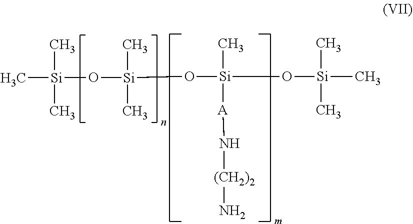

in which:
m and n are numbers such that the sum (n+m) ranges from 1 to 2000 and in particular from 50 to 150, n possibly denoting a number from 0 to 1999 and notably from 49 to 149, and m possibly denoting a number from 1 to 2000 and notably from 1 to 10; and
A denotes a linear or branched alkylene radical containing from 4 to 8 carbon atoms and preferably 4 carbon atoms. This radical is preferably branched.

The weight-average molecular mass (Mw) of these aminosilicones preferably ranges from 500 to 1 000 000 g/mol and more particularly from 1000 to 200 000 g/mol. A silicone corresponding to this formula is, for example, DC2-8566 Amino Fluid from Dow Corning;

c) the aminosilicones corresponding to formula (VIII):

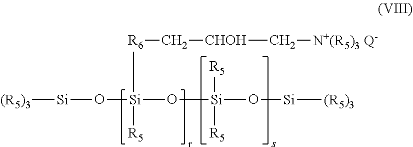

in which:
R₅ represents a monovalent hydrocarbon-based radical containing from 1 to 18 carbon atoms, and in particular a C₁-C₁₈ alkyl or C₂-C₁₈ alkenyl, for example methyl, radical;
R₆ represents a divalent hydrocarbon radical, notably a C₁-C₁₈ alkylene radical or a divalent C₁-C₁₈, for example C₁-C₈, alkyleneoxy radical connected to the Si via an SiC bond;
Q⁻ is an anion such as a halide ion, notably chloride, or an organic acid salt, notably acetate;
r represents a mean statistical value ranging from 2 to 20 and in particular from 2 to 8; and
s represents a mean statistical value ranging from 20 to 200 and in particular from 20 to 50.

Such aminosilicones are notably described in patent U.S. Pat. No. 4,185,087.

d) the quaternary ammonium silicones of formula (IX):

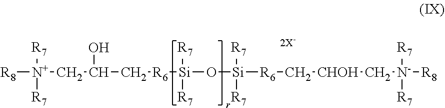

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon radical, notably a C₁-C₁₈ alkylene radical or a divalent C₁-C₁₈, for example C₁-C₈, alkyleneoxy radical connected to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl substituent or a —R₆-NHCOR₇ radical;
X⁻ is an anion such as a halide ion, notably chloride, or an organic acid salt, notably acetate; and
r represents a mean statistical value ranging from 2 to 200 and in particular from 5 to 100.

Such aminosilicones are notably described in patent application EP-A 0 530 974.

e) the aminosilicones of formula (X):

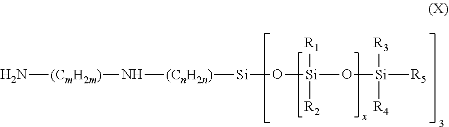

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;

f) multiblock polyoxyalkylenated aminosilicones, of the type (AB)ₙ, A being a polysiloxane block and B being a polyoxyalkylenated block including at least one amine group.

Said silicones are preferably formed from repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which may be identical or different, represent a linear or branched divalent C₂-C₁₂ hydrocarbon-based radical, optionally including one or more heteroatoms such as oxygen; preferably, R, which may be identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which may be identical or different, represent a linear or branched divalent C₂-C₁₂ hydrocarbon-based radical, optionally including one or more heteroatoms such as oxygen; preferably, R′, which may be identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.

The siloxane blocks preferably represent 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 g/mol and more particularly between 10 000 and 200 000 g/mol.

Mention may notably be made of the silicones sold under the name Silsoft A-843 or Silsoft A+ by Momentive.

g) the aminosilicones of formulae (XI) and (XII):

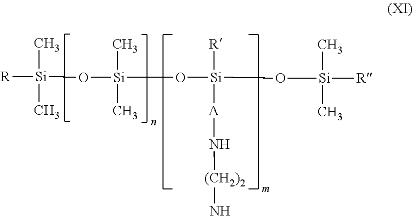

in which:
R, R′ and R″, which may be identical or different, denote a C₁-C₄ alkyl group or a hydroxyl group,
A denotes a C₃ alkylene radical; and
m and n are numbers such that the weight-average molecular mass of the compound is between 5000 and 500 000

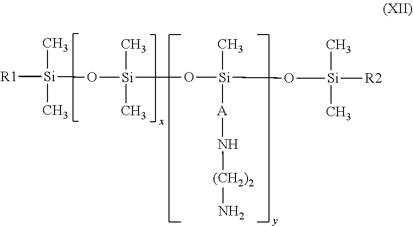

in which:
x and y are numbers ranging from 1 to 5000; preferably, x ranges from 10 to 2000 and more preferentially from 100 to 1000; preferably, y ranges from 1 to 100;
R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.

Preferably, A comprises from 3 to 6 carbon atoms, more preferentially 4 carbon atoms; preferably, A is branched.

Mention may be made in particular of the following divalent groups: —CH₂CH₂CH₂— and —CH₂CH(CH₃)CH₂—.

Preferably, R₁ and R₂ are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which may be identical or different, are chosen from hexadecyl (cetyl) and octadecyl (stearyl) groups.

The aminosilicone(s) are preferably of formula (XII) with:
x ranging from 10 to 2000 and in particular from 100 to 1000;
y ranging from 1 to 100;
A comprising from 3 to 6 carbon atoms and notably 4 carbon atoms;
preferably, A is branched; more particularly, A is chosen from the following divalent groups: —CH₂CH₂CH₂— and —CH₂CH(CH₃)CH₂—; and
R₁ and R₂ independently being saturated linear alkyl groups comprising from 6 to 30 carbon atoms, preferably from 8 to 24 carbon atoms and in particular from 12 to 20 carbon atoms; chosen notably from dodecyl, tetradecyl, pentadecyl, hexadecyl, heptadecyl, octadecyl, nonadecyl and eicosyl groups; preferentially, R₁ and R₂, which may be identical or different, are chosen from hexadecyl (cetyl) and octadecyl (stearyl) groups.

A silicone of formula (XII) that is preferred is bis-cetearyl amodimethicone. Mention may be made in particular of the aminosilicone sold under the name Silsoft AX by Momentive.

h) polysiloxanes and notably polydimethylsiloxanes, including primary amine groups at only one chain end or on side chains, such as those of formula (XIV), (XV) or (XVI):

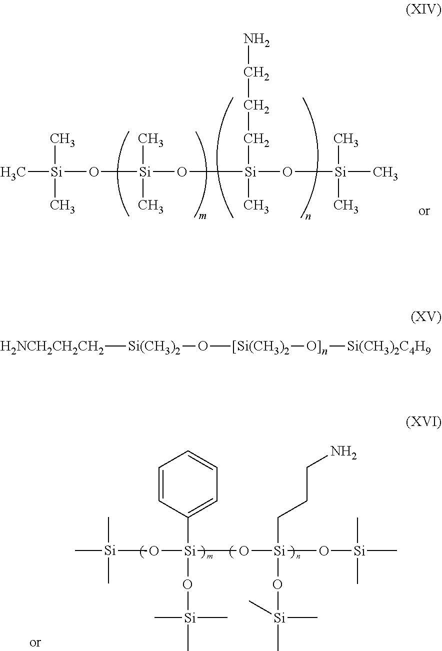

In formula (XIV), the values of n and m are such that the weight-average molecular mass of the aminosilicone is between 1000 and 55 000.

As examples of aminosilicones of formula (XIV), mention may be made of the products sold under the names AMS-132, AMS-152, AMS-162, AMS-163, AMS-191 and AMS-1203 by the company Gelest and KF-8015 by the company Shin-Etsu.

In formula (XV), the values of n and m are such that the weight-average molecular mass of the aminosilicone is between 500 and 3000.

As examples of aminosilicones of formula (XV), mention may be made of the products sold under the names MCR-A11 and MCR-A12 by the company Gelest. In formula (XVI), the values of n and m are such that the weight-average molecular mass of the aminosilicone is between 500 and 50 000.

As examples of aminosilicones of formula (XVI), mention may be made of the aminopropyl phenyl trimethicone sold under the name DC 2-2078 Fluid by the company Dow Corning.

i) and mixtures thereof.

Preferably, the aminosilicone(s) (b) are chosen from the aminosilicones of formulae (II), (XII) and (XVI) and mixtures thereof, more preferentially chosen from the aminosilicones of formula (XII), and mixtures thereof, and better still the aminosilicone (b) is bis-cetearyl amodimethicone.

The content of the aminosilicone(s) (b) present in the composition according to the present invention preferably ranges from 0.05% to 10% by weight, preferentially from 0.1% to 5% by weight, and more preferentially from 0.2% to 2% by weight, relative to the total weight of the composition.

The weight ratio (Ra) between the total content of said hydrophobic film-forming polymer(s) (a) and the total content of the aminosilicone(s) (b) is preferably greater than or equal to 1, more preferentially greater than or equal to 2, and better still this weight ratio (Ra) ranges from 5 to 15.

In another embodiment of the invention, when the composition comprises one or more hydrocarbon-based block copolymers (a′), the weight ratio (Ra′) between the total content of said hydrocarbon-based block copolymer(s) (a′) and the content of the aminosilicone(s) (b) is preferably greater than or equal to 1, more preferentially greater than or equal to 2, and better still this weight ratio (Ra′) ranges from 5 to 15.

The Organic Solvents

The composition according to the present invention also comprises (c) one or more organic solvents.

The organic solvent(s) according to the invention may be chosen from C₁ to C₄ alcohols, such as ethanol or isopropanol, polyols and polyol ethers such as glycerol, 2-butoxyethanol, propylene glycol, hexylene glycol, dipropylene glycol, propylene glycol monomethyl ether, propylene glycol monoethyl ether, aromatic alcohols or ethers, such as benzyl alcohol and phenoxyethanol, hydrocarbon-based oils, and mixtures thereof.

Preferably, the organic solvent(s) (c) are chosen from hydrocarbon-based oils. In other words, the composition according to the invention comprises one or more hydrocarbon-based oils.

This oil may be volatile (vapour pressure greater than or equal to 0.13 Pa measured at 25° C.) or non-volatile (vapour pressure less than 0.13 Pa measured at 25° C.).

Preferably, the hydrocarbon-based oil is volatile.

The hydrocarbon-based oil is an oil (non-aqueous compound) that is liquid at room temperature (25° C.) and at atmospheric pressure.

The term “hydrocarbon-based oil” means an oil formed essentially from, or even constituted of, carbon and hydrogen atoms, and optionally oxygen and nitrogen atoms, and not containing any silicon or fluorine atoms. It may contain alcohol, ester, ether, carboxylic acid, amine and/or amide groups.

The term “hydrocarbon” means a compound formed solely from carbon and hydrogen atoms.

The hydrocarbon-based oil is preferably chosen from:
branched C₅-C₁₆ alkanes such as C₅-C₁₆ isoalkanes of petroleum origin (also known as isoparaffins), for instance isododecane (also known as 2,2,4,4,6-pentamethylheptane), isodecane, isohexadecane and, for example, the oils sold under the trade name Isopar or Permethyl;
linear C₈ to C₁₆ alkanes, for instance n-dodecane (C₁₂) and n-tetradecane (C₁₄) sold by Sasol under the references, respectively, Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155059 from the company Cognis, and mixtures thereof;
short-chain esters (containing from 3 to 8 carbon atoms in total) such as ethyl acetate, methyl acetate, propyl acetate or n-butyl acetate;
hydrocarbon-based oils of plant origin such as triglycerides consisting of fatty acid esters of glycerol, the fatty acids of which may have chain lengths ranging from C₄ to C₂₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stéarinerie Dubois or those sold under the names Miglyol 810, 812 and 818 by the company Dynamit Nobel;
synthetic ethers containing from 10 to 40 carbon atoms;
linear or branched hydrocarbons of mineral or synthetic origin comprising more than 16 carbon atoms, such as petroleum jelly, polydecenes, hydrogenated polyisobutene such as Parleam®, squalane and liquid paraffins, and mixtures thereof;
synthetic esters such as oils of formula R₁COOR₂ in which R₁ represents a linear or branched fatty acid residue including from 1 to 40 carbon atoms and R₂ represents a, notably branched, hydrocarbon-based chain containing from 1 to 40 carbon atoms, on condition that R₁+R₂≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters;
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol; and
mixtures thereof.

Advantageously, the hydrocarbon-based oil is apolar (thus formed solely from carbon and hydrogen atoms), and better still it is chosen from hydrocarbons.

The hydrocarbon-based oil is preferably chosen from hydrocarbon-based oils containing from 8 to 16 carbon atoms, more preferentially from 10 to 16 carbon atoms, and better still apolar oils.

Preferentially, the hydrocarbon-based oil is chosen from C₈ to C₁₆ and preferably C₁₀ to C₁₆ linear or branched alkanes, more preferentially chosen from C₈ to C₁₆ and preferably C₁₀ to C₁₆ branched alkanes. Particularly preferably, the hydrocarbon-based oil is chosen from isododecane, undecane and tridecane, and mixtures thereof. More preferentially, the composition according to the invention comprises isododecane.

Preferably, when the composition comprises one or more hydrocarbon-based oils, these oils constitute the only oil(s) of the composition, or are present in a predominant content relative to the additional oils that may be present in the composition.

The total content of the organic solvent(s) (c), present in the composition according to the invention, preferably ranges from 10% to 99% by weight, preferentially from 70% to 98% by weight, and more preferentially from 80% to 95% by weight relative to the total weight of the composition.

The composition according to the invention may be aqueous or anhydrous.

In a preferred embodiment, the composition is anhydrous. For the purposes of the present invention, the term “anhydrous composition” means a composition comprising a water content of less than or equal to 1% by weight, preferably less than or equal to 0.5% by weight, even more preferentially less than or equal to 0.1% by weight, relative to the total weight of the composition. Better still, the anhydrous composition according to the invention is totally free of water (0%).

In another preferred embodiment, the composition is aqueous. For the purposes of the present invention, the term “aqueous composition” means a composition comprising water in a content preferably ranging from 50% to 99% by weight, more preferentially from 60% to 95% by weight, even more preferentially from 65% to 90% by weight, relative to the total weight of the composition.

In one particular embodiment of the invention, the composition may comprise at least two distinct phases, and may notably be in the form of a double phase, notably of a double phase comprising an aqueous phase and an anhydrous phase, the two distinct phases preferably being one on top of the other; in particular, the aqueous phase is below the anhydrous phase.

The Phenyl Silicones

The composition according to the present invention may optionally also comprise one or more silicones other than the aminosilicones (b) and comprising one or more optionally substituted phenyl groups in their structure, i.e. one or more phenyl silicones.

According to a preferred embodiment, the composition according to the invention also comprises one or more silicones other than the aminosilicones (b) and comprising one or more optionally substituted phenyl groups in their structure.

Preferably, the silicone(s) other than the aminosilicones (b) and comprising one or more phenyl groups are non-volatile; they are preferentially chosen from silicones comprising two phenyl groups, silicones comprising three phenyl groups, silicones comprising four phenyl groups, silicones comprising five phenyl groups, silicones comprising six phenyl groups, and mixtures thereof.

A silicone comprising one or more phenyl groups that is particularly preferred is pentaphenyl dimethicone, al so known under the name trimethylpentaphenyltrisiloxane, such as the product sold by Dow Corning under the trade name Dow Corning PH-1555 HRI Cosmetic Fluid®.

Other silicones comprising one or more phenyl groups that may be used are phenyl trimethicones and polyphenyl trimethicones preferably having molecular weights of at least 3000 Da and/or viscosities of at least 100 cSt, such as:
the phenyl trimethicone sold in particular by Evonik Goldschmidt under the trade names Abil AV 350 (300±30 cSt) and Abil AV 1000 (925-1075 cSt) and also commercially available from Dow Corning under the brand name Dow Corning DC1-0648 (at least 5000 cSt),
the trimethylsiloxyphenyl dimethicone sold in particular by the company Wacker under the brand name Wacker-Belsil PDM 1000 (925-1075 cSt), and
the diphenylsiloxyphenyl trimethicone sold in particular by the company Faconnier.

The total content of the silicone(s) other than the aminosilicones (b) and comprising one or more phenyl groups, when they are present in the composition according to the invention, preferably ranges from 0.1% to 10% by weight, preferentially from 0.2% to 7% by weight, and more preferentially from 0.5% to 2% by weight, relative to the total weight of the composition.

The weight ratio (Rb) between the total content of said hydrophobic film-forming polymer(s) (a) and the total content of the silicone(s) other than the aminosilicone(s) (b) and comprising one or more phenyl groups is preferably greater than or equal to 1, more preferentially greater than or equal to 2, and better still this weight ratio (Rb) ranges from 2 to 10 and even more preferentially from 2 to 5.

In another embodiment of the invention, when the composition comprises one or more hydrocarbon-based block copolymers (a′), the weight ratio (Rb′) between the total content of said hydrocarbon-based block copolymer(s) (a′) and the total content of the silicone(s) other than the aminosilicone(s) (b) and comprising one or more phenyl groups is preferably greater than or equal to 1, more preferentially greater than or equal to 2, and better still this weight ratio (Rb′) ranges from 2 to 10 and even more preferentially from 2 to 5.

The Dyestuffs

The composition according to the present invention may optionally also comprise one or more dyestuffs, preferably chosen from pigments.

The term “pigment” is intended to denote a white or coloured solid particle which is naturally insoluble in the hydrophilic and lipophilic liquid phases usually employed in cosmetics or which is rendered insoluble by formulation in the form of a lake, where appropriate. More particularly, the pigment has little or no solubility in aqueous-alcoholic media.

Pigments that may be mentioned include organic and mineral pigments such as those defined and described in Ullmann's Encyclopedia of Industrial Chemistry “Pigment organics”, 2005 Wiley-VCH Verlag GmbH & Co. KGaA, Weinheim 10.1002/14356007.a20 371 and ibid, “Pigments, Inorganic, 1. General” 2009 Wiley-VCH Verlag GmbH & Co. KGaA, Weinheim10.1002/14356007.a20 243.pub3.

Mention may be made notably of azo pigments which contain one or more azo groups A-N═N—B with A representing an optionally substituted (hetero)aryl, B representing optionally substituted (hetero)aryl or —CH[C(O)—R]—C(O)—X₁-A′, A′ representing an optionally substituted (hetero)aryl and R representing a hydrogen atom or a (C₁-C₆)alkyl group, with the (hetero)aryl groups A, A′ and B not containing any solubilizing groups such as —SO₃H or —COOH.

They may in particular be monoazo pigments, including β-naphthols, monoazopyrrolone pigments or benzimidazolone pigments; diazo pigments, such as diazodiarylide pigments and bis(N-acetoacetarylide) pigments, or triazo or tetraazo pigments.

Mention may also be made of azo metal complex pigments.

Other pigments are also advantageous, namely isoindolinone and isoindoline pigments, phthalocyanine pigments; quinacridone pigments; perinone pigments; perylene pigments; anthraquinone pigments such as hydroxyanthraquinone pigments; ami noanthraqui none pigments including acyl ami noanthraqui none s and azo anthraquinone pigments; heterocyclic anthraquinones; polycarbocyclic anthraquinone pigments, pyranthrone pigments; anthranthrone pigments; diketopyrrolopyrrole (DPP) pigments; thioindigo pigments; dioxazine pigments; triphenylmethane pigments; quinophthalone pigments; and fluorescent pigments.

When the dyes comprise one or more solubilizing groups such as —SO₃H or —COOH, these dyes are made insoluble and consequently pigments by formation of a lake, i.e. by salification (e.g. Na, Ca, St, Ba, etc.) and divided mainly into β-naphthol and 2-hydroxy-3-naphthoic acid pigments “(BON) pigment lakes”.

In the context of the present invention, the pigment may be at least partly organic.

According to one embodiment of the invention, the pigment is an organic pigment.

According to another embodiment of the invention, the pigment is a mineral pigment.

The microcapsules according to the invention comprise not more than 80% by weight of pigment relative to the weight of the polymer matrix. In particular, they may comprise from 0.5% to 75% by weight, for example from 1% to 70% by weight, notably from 20% to 65% by weight or even from 30% to 60% by weight of pigment relative to the weight of the polymer matrix.

Needless to say, the degree of encapsulation depends on the desired modification of the shade and may thus vary significantly according to the effect that it is desired to obtain.

As illustrations of pigments that may be used in the present invention, mention may be made of carbon black, titanium oxide, chromium oxide, pigments of D&C or FD&C type and lakes thereof, and notably those known under the names D&C Blue No. 4, D&C Brown No. 1, FD&C Green No. 3, D&C Green No. 5, D&C Green No. 6, FD&C Green No. 8, D&C Orange No. 4, D&C Orange No. 5, D&C Orange No. 10, D&C Orange No. 11, FD&C Red No. 4, D&C Red No. 6, D&C Red No. 7 (CI 15850), D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, FD&C Red 40 lake, D&C Violet No. 2, Ext. D&C Violet No. 2, FD & C Blue No. 1, D&C Yellow No. 6, FD&C Yellow No. 6, D&C Yellow No. 7, 25 Ext. D&C Yellow No. 7, D&C Yellow No. 8, D&C Yellow No. 10 or D&C Yellow No. 11, it being understood that when said pigment is not naturally insoluble in the hydrophilic and lipophilic phases usually used in cosmetics, it is used in the form of a corresponding lake, as explained previously.

Examples of lakes that may notably be mentioned include lakes based on barium, strontium, calcium or aluminium, or alternatively diketopyrrolopyrroles.

As further examples of pigments that may be used in the present invention, mention may be made notably of mineral pigments, optionally surface-treated and/or coated, and notably titanium dioxide, zirconium oxide or cerium oxide, and also zinc oxide, iron oxide (black, yellow or red) or chromium oxide, manganese violet, ultramarine blue, chromium hydrate and ferric blue, or alternatively metal powders, for instance aluminium powder, copper powder, gold powder and silver powder.

Mention may also be made of pigments with an optical effect such as particles including a natural or synthetic organic or mineral substrate, for example glass, acrylic resins, polyester, polyurethane, polyethylene terephthalate, ceramics or aluminas, said substrate being optionally covered with metal substances, for instance aluminium, gold, silver, platinum, copper or bronze, or with metal oxides, for instance titanium dioxide, iron oxide or chromium oxide.

They may also be nacres.

The term “nacres” means iridescent pigments, which are notably produced by certain molluscs in their shell, or alternatively which are synthesized.

The nacreous pigments may be chosen from mica coated with titanium or with bismuth oxychloride, titanium mica coated with iron oxides, titanium mica coated notably with ferric blue or with chromium oxide, titanium mica coated with an organic pigment of the abovementioned type, and also nacreous pigments based on bismuth oxychloride. Use may also be made of interference pigments, notably liquid-crystal or multilayer pigments.

They may also be pigments having a structure that may be, for example, of sericite/brown iron oxide/titanium dioxide/silica type.

They may also be pigments having a structure that may be, for example, of silica microsphere type containing iron oxide.

As examples of pigments that are most particularly suitable for use in the present invention, mention may be made notably of D&C Red No. 7, titanium oxide, chromium oxide, lakes of the pigments of D&C and FD&C type mentioned above, and notably D&C Red No. 22 lake, Yellow No. 6 lake and FD&C Blue No. 1 lake.

The pigments in accordance with the invention may be in the form of pigment powder or paste. They may be coated or uncoated.

The pigments in accordance with the invention may be chosen, for example, from white or coloured pigments, lakes, pigments with special effects such as nacres or glitter flakes, and mixtures thereof.

Examples of white or coloured mineral pigments that may be mentioned include zirconium oxide or cerium oxide, chromium oxides, manganese violet, ultramarine blue, chromium hydrate and ferric blue.

Examples of white or coloured organic pigments that may be mentioned include nitroso, nitro, azo, xanthene, quinoline, anthraquinone and phthalocyanine compounds, compounds of metallic complex type, and isoindolinone, isoindoline, quinacridone, perinone, perylene, diketopyrrolopyrrole, thioindigo, dioxazine, triphenylmethane and quinophthalone compounds.

In particular, the white or coloured organic pigments may be chosen from carmine, carbon blacks such as Black 2, aniline black, azo yellow, quinacridone, phthalocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in patent FR 2 679 771.

Preferably, carbon blacks such as Black 2 or lakes such as D&C Red 7 are chosen.

Use may be made of pigment pastes of organic pigments, such as the products sold by the company Hoechst under the names:
Cosmenyl Yellow IOG: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including:
a mineral core,
at least one binder for fixing the organic pigments to the core, and
at least one organic pigment at least partially covering the core.

The term “lake” refers to dyes adsorbed onto insoluble particles, the assembly thus obtained remaining insoluble during use. The mineral substrates onto which the dyes are adsorbed are, for example, alumina, silica, calcium sodium borosilicate or calcium aluminium borosilicate and aluminium. Among the organic dyes, mention may be made of cochineal carmine.

Examples of lakes that may be mentioned include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053) or D & C Blue 1 (CI 42 090).

The term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from white or coloured pigments that afford a standard uniform opaque, semi-transparent or transparent shade.

Examples of pigments with special effects that may be mentioned include white nacreous pigments such as mica coated with titanium or with bismuth oxychloride, coloured nacreous pigments such as mica coated with titanium and with iron oxides, mica coated with titanium and notably with ferric blue or with chromium oxide, mica coated with titanium and with an organic pigment as defined previously, and also nacreous pigments based on bismuth oxychloride.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductor nanoparticles that are capable of emitting, under light excitation, radiation with a wavelength of between 400 nm and 700 nm. These nanoparticles are known from the literature. In particular, they may be manufactured according to the processes described, for example, in U.S. Pat. No. 6,225,198 or

U.S. Pat. No. 5,990,479, in the publications cited therein and also in the following publications: Dabboussi B. O. et al., “(CdSe)ZnS core-shell quantum dots: synthesis and characterisation of a size series of highly luminescent nanocrystallites”, Journal of Physical Chemistry B, vol. 101, 1997, pages 9463-9475, and Peng, Xiaogang et al., “Epitaxial growth of highly luminescent CdSe/CdS core/shell nanocrystals with photostability and electronic accessibility”, Journal of the American Chemical Society, vol. 119, No. 30, pages 7019-7029.

The pigments in accordance with the invention are preferably coloured pigments.

The variety of pigments used makes it possible to obtain a wide range of colours, and also particular optical effects such as metallic or interference effects.

The size of a pigment other than the nacres in solution is generally between 10 nm and 10 μm, preferably between 50 nm and 5 μm and even more preferentially between 100 nm and 3 μm. The size of a nacre in solution is generally between 1 and 200 μm, preferably between 1 and 80 μm and even more preferentially between 1 and 50 μm.

Among the mineral pigments, examples that may be mentioned include titanium dioxide (rutile or anatase) optionally surface-treated and codified in the Color Index under the reference CI 77891; black, yellow, red and brown iron oxides, codified under the references CI 77499, 77492 and 77491; manganese violet (CI 77742); ultramarine blue (CI 77007); hydrated chromium oxide (CI 77289); ferric blue (CI77510).

Among the organic pigments that may be mentioned, for example, are the pigment Yellow 3 sold notably under the trade name Jaune Covanor W 1603 by the company Wackherr (CI 17710), D & C Red No. 19 (CI 45170), D & C Red No. 9 (CI 15585), D & C Red No. 21 (CI 45380), D & C Orange No. 4 (CI 15510), D & C Orange No. 5 (CI 45370), D & C Red No. 27 (CI 45410), D & C Red No. 13 (CI 15630), D & C Red No. 7 (CI 15850-1), D & C Red No. 6 (CI 15850-2), D & C Yellow No. 5 (CI 19140), D & C Red No. 36 (CI 12085), D & C Orange No. 10 (CI 45425), D & C Yellow No. 6 (CI 15985), D & C Red No. 30 (CI 73360), D & C Red No. 3 (CI 45430), carbon black (CI 77266) and lakes based on cochineal carmine (CI 75470).

It is also possible to use nacreous pigments, which may be chosen notably from white nacreous pigments such as mica coated with titanium oxide or bismuth oxide; coloured nacreous pigments such as titanium mica with iron oxides, titanium mica with ferric blue or with chromium oxide, titanium mica with an organic pigment of the abovementioned type, and also those based on bismuth oxychloride.

Pigment pastes of organic pigment are used more particularly, such as the products sold by the company Hoechst under the names:
Cosmenyl Yellow 10G: Yellow 3 pigment (CI 11710),
Cosmenyl Yellow G: Yellow 1 pigment (CI 11680),
Cosmenyl Orange GR: Orange 43 pigment (CI 71105),
Cosmenyl Red R: Red 4 pigment (CI 12085),
Cosmenyl Carmine FB: Red 5 pigment (CI 12490),
Cosmenyl Violet RL: Violet 23 pigment (CI 51319)
Cosmenyl Blue A2R: Blue 15.1 pigment (CI 74260)
Cosmenyl Green GG: Green 7 pigment (CI 74260)
Cosmenyl Black R: Black 7 pigment (CI 77266).

The dyestuff(s) are advantageously chosen from pigments and mixtures thereof, and preferably from nacres and mixtures thereof.

The total content of dyestuff(s), if present in the composition according to the present invention, preferably ranges from 0.1% to 15% by weight, more preferentially from 0.5% to 10% by weight, and better still from 1% to 5% by weight, relative to the total weight of the composition.

The Polymer Particles

The composition according to the present invention may also optionally comprise particles, which are preferably spherical, of at least one surface-stabilized polymer.

Preferably, the particles are introduced into the composition in the form of a dispersion of particles, which are generally spherical, of at least one surface-stabilized polymer, in an oily medium, advantageously containing at least one hydrocarbon-based oil, as defined previously.

The polymer of the particles is preferably a polymer obtained from monomers chosen from C₁-C₄ alkyl (meth)acrylates. It may be a homopolymer or a copolymer obtained by polymerization of only one or of several different monomers.

The C₁-C₄ alkyl (meth)acrylate monomers may be chosen from methyl (meth)acrylate, ethyl (meth)acrylate, n-propyl (meth)acrylate, isopropyl (meth)acrylate, n-butyl (meth)acrylate and tert-butyl (meth)acrylate.

A C₁-C₄ alkyl acrylate monomer is advantageously used. Preferentially, the polymer of the particles is obtained from methyl acrylate and/or ethyl acrylate.

The polymer of the particles may also comprise an ethylenically unsaturated acid monomer or an anhydride thereof, chosen notably from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof.

The ethylenically unsaturated acid monomer is preferably chosen from (meth)acrylic acid, maleic acid and maleic anhydride.

The salts may be chosen from salts of alkali metals, for example sodium or potassium; salts of alkaline-earth metals, for example calcium, magnesium or strontium; metal salts, for example zinc, aluminium, manganese or copper; ammonium salts of formula NH₄⁺; quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles may thus comprise or consist essentially of from 80% to 100% by weight of C₁ to C₄ alkyl (meth)acrylate and from 0 to 20% by weight of ethylenically unsaturated acid monomer, relative to the total weight of the polymer.

According to a first embodiment of the invention, the polymer consists essentially of a polymer obtained from one or more monomers chosen from C₁ to C₄ alkyl (meth)acrylate(s).

According to a second embodiment of the invention, the polymer consists essentially of a copolymer of C₁ to C₄ (meth)acrylate(s) and of (meth)acrylic acid or maleic anhydride.

The polymer of the particles may be chosen from:
methyl acrylate homopolymers,
ethyl acrylate homopolymers,
methyl acrylate/ethyl acrylate copolymers,
methyl acrylate/ethyl acrylate/acrylic acid copolymers,
methyl acrylate/ethyl acrylate/maleic anhydride copolymers,
methyl acrylate/acrylic acid copolymers,
ethyl acrylate/acrylic acid copolymers,
methyl acrylate/maleic anhydride copolymers, and
ethyl acrylate/maleic anhydride copolymers.

The polymer of the particles may also be a polymer obtained from monomers chosen from aryl (meth)acrylates, and more particularly benzyl acrylate.

Advantageously, the polymer of the particles is a non-crosslinked polymer.

The polymer of the particles preferably has a number-average molecular weight (Mn) ranging from 2000 to 10 000 000 and preferably ranging from 150 000 to 500 000.

In the case of a particle dispersion, the polymer(s) of the particles may preferably be present in the dispersion in a content ranging from 20% to 60% by weight, more preferentially from 21% to 58.5% by weight, better still from 30% to 50% by weight and even better still from 36% to 42% by weight, relative to the total weight of the dispersion.

The polymer is surface-stabilized, for example using a stabilizer which is preferably a homopolymer or a copolymer obtained notably from isobornyl (meth)acrylate(s), and better still chosen from isobornyl (meth)acrylate homopolymer and statistical copolymers of isobornyl (meth)acrylate and of C₁-C₄ alkyl (meth)acrylate present in an isobornyl (meth)acrylate/C₁-C₄ alkyl (meth)acrylate weight ratio of greater than 4, preferably greater than 4.5 and even more advantageously greater than or equal to 5. Advantageously, said weight ratio ranges from 4.5 to 19, preferably from 5 to 19 and more particularly from 5 to 12.

According to a particular embodiment, the stabilizer is a statistical copolymer of isobornyl (meth)acrylate and of C₁ to C₄ alkyl (meth)acrylate present in an isobornyl (meth)acrylate/C₁ to C₄ alkyl (meth)acrylate weight ratio of greater than or equal to 5.

Advantageously, the stabilizer is chosen from:
isobornyl acrylate homopolymers,
statistical copolymers of isobornyl acrylate/methyl acrylate,
statistical copolymers of isobornyl acrylate/methyl acrylate/ethyl acrylate, and
statistical copolymers of isobornyl methacrylate/methyl acrylate, in the weight ratio described previously.

The stabilizer preferably has a number-average molecular weight (Mn) ranging from 10 000 to 400 000 and preferably ranging from 20 000 to 200 000.

The stabilizer is in contact with the surface of the polymer particles and thus makes it possible to stabilize these particles at the surface, in particular in order to keep these particles in dispersion in the non-aqueous medium of the dispersion.

Advantageously, the combination of the stabilizer(s)+polymer(s) of the particles present in particular in the dispersion comprises in total from 10% to 50% by weight of polymerized isobornyl (meth)acrylate and from 50% to 90% by weight of polymerized C₁ to C₄ alkyl (meth)acrylate, relative to the total weight of the combination of the stabilizer(s)+polymer(s) of the particles.

Preferentially, the combination of the stabilizer(s)+polymer(s) of the particles present in particular in the dispersion comprises in total from 15% to 30% by weight of polymerized isobornyl (meth)acrylate and from 70% to 85% by weight of polymerized C₁ to C₄ alkyl (meth)acrylate, relative to the total weight of the combination of the stabilizer(s)+polymer(s) of the particles.

Preferably, the stabilizer(s) are soluble in the hydrocarbon-based oils as defined previously, and in particular soluble in isododecane.

When the polymer particles are provided in the composition in the form of a dispersion prepared beforehand, the oily medium of this polymer dispersion comprises at least one hydrocarbon-based oil as defined previously.

Advantageously, the hydrocarbon-based oil is apolar and preferably chosen from hydrocarbon-based oils containing from 8 to 16 carbon atoms, in particular the apolar oils described previously.

Preferentially, the hydrocarbon-based oil is isododecane.

The polymer particles, in particular in the dispersion, preferably have a mean size, notably a number-mean size, ranging from 50 to 500 nm, notably ranging from 75 to 400 nm and better still ranging from 100 to 250 nm.

In general, a dispersion of polymer particles that is suitable for use in the invention may be prepared in the following manner, which is given as an example.

The polymerization may be performed in dispersion, i.e. by precipitation of the polymer during formation, with protection of the formed particles with a stabilizer.

In a first step, the stabilizing polymer is prepared by mixing the constituent monomer(s) of the stabilizing polymer with a free-radical initiator, in a solvent known as the synthesis solvent, and by polymerizing these monomers. In a second step, the constituent monomer(s) of the polymer of the particles are added to the stabilizing polymer formed and polymerization of these added monomers is performed in the presence of the free-radical initiator.

When the non-aqueous medium is a non-volatile hydrocarbon-based oil, the polymerization may be performed in an apolar organic solvent (synthesis solvent), followed by adding the non-volatile hydrocarbon-based oil (which should be miscible with said synthesis solvent) and selectively distilling off the synthesis solvent.

A synthesis solvent which is such that the monomers of the stabilizing polymer and the free-radical initiator are soluble therein, and the polymer particles obtained are insoluble therein, so that they precipitate therein during their formation, is thus chosen.

In particular, the synthesis solvent may be chosen from alkanes such as heptane or cyclohexane.

When the non-aqueous medium is a volatile hydrocarbon-based oil, the polymerization may be performed directly in said oil, which thus also acts as synthesis solvent. The monomers should also be soluble therein, as should the free-radical initiator, and the polymer of the particles which is obtained should be insoluble therein.

The monomers are preferably present in the synthesis solvent, before polymerization, in a proportion of 5% to 20% by weight. The total amount of the monomers may be present in the solvent before the start of the reaction, or part of the monomers may be added gradually as the polymerization reaction proceeds.

The free-radical initiator may notably be azobisisobutyronitrile or tert-butyl peroxy-2-ethylhexanoate.

The polymerization may be performed at a temperature ranging from 70 to 110° C.

The polymer particles are surface-stabilized, when they are formed during the polymerization, by means of the stabilizer.

The stabilization may be performed by any known means, and in particular by direct addition of the stabilizer, during the polymerization.

The stabilizer is preferably also present in the mixture before polymerization of the monomers of the polymer of the particles. However, it is also possible to add it continuously, notably when the monomers of the polymer of the particles are also added continuously.

From 10% to 30% by weight of stabilizer may be used relative to the total weight of monomers used.

The Additional Non-Amino Silicones

The composition according to the present invention may also optionally comprise one or more additional non-amino and non-phenyl silicones.

The term “non-amino silicone” means any silicone which does not include any primary, secondary, tertiary or quaternary amine groups, namely any silicone other than the aminosilicones (b) mentioned above.

The additional non-amino, non-phenyl silicones that may be used in the composition according to the invention are in particular polyorganosiloxanes, which may be in the form of aqueous solutions, i.e. dissolved, or optionally in the form of dispersions or microdispersions, or of aqueous emulsions. The polyorganosiloxanes may also be in the form of oils, waxes or gums.

Organopolysiloxanes are defined in greater detail in Walter Noll's Chemistry and Technology of Silicones (1968), Academic Press.

The additional non-amino, non-phenyl silicones may be volatile or non-volatile.

When they are volatile, the non-amino silicones are more particularly chosen from those with a boiling point between of 60° C. and 260° C., and even more particularly from:

(i) cyclic silicones including from 3 to 7 and preferably 4 to 5 silicon atoms.

These are, for example, octamethylcyclotetrasiloxane sold notably under the name Volatile Silicone 7207 by the company Union Carbide or Silbione 70045 V 2 by the company Rhodia, decamethylcyclopentasiloxane sold under the name Volatile Silicone 7158 by the company Union Carbide, and Silbione 70045 V 5 by the company Rhodia, and mixtures thereof.

Mention may also be made of cyclocopolymers of the dimethylsiloxane/methylalkylsiloxane type, such as Volatile Silicone FZ 3109 sold by the company Union Carbide, of chemical structure:

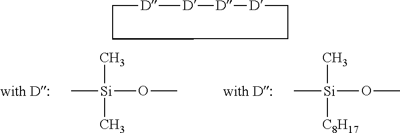

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis[2,2,2′,2′,3,3′-hexa(trimethylsilyloxy)neopentane];

(ii) linear volatile silicones containing 2 to 9 silicon atoms and having a viscosity of less than or equal to 5×10⁻⁶ m²/s at 25° C. An example is decamethyltetrasiloxane sold notably under the name SH 200 by the company Toray Silicone. Silicones falling within this category are also described in the article published in Cosmetics and Toiletries, Vol. 91, January 76, pages 27-32—Todd & Byers Volatile Silicone Fluids for Cosmetics.

When the non-amino, non-phenyl silicones are non-volatile, use is preferably made of polyalkylsiloxanes, silicone gums, and polyorganosiloxanes modified with organofunctional groups, and mixtures thereof.

These non-amino silicones are more particularly chosen from polyalkylsiloxanes, among which mention may be made mainly of polydimethylsiloxanes containing trimethylsilyl end groups (Dimethicone according to the CTFA name) having a viscosity of from 5×10⁻⁶ to 2.5 m²/s at 25° C. and preferably 1 ×10⁻⁵ to 1 m²/s. The viscosity of the silicones is measured, for example, at 25° C. according to standard ASTM 445 Appendix C.

Among these polyalkylsiloxanes, mention may be made, in a non-limiting manner, of the following commercial products:
the Silbione oils of the 47 and 70 047 series or the Mirasil oils sold by the company Rhodia, for instance the oil 70 047 V 500 000,
the oils of the Mirasil series sold by the company Rhodia,
the oils of the 200 series from the company Dow Corning, such as, more particularly, DC200 with a viscosity of 60 000 cSt,
the Viscasil oils from the company General Electric and certain oils of the SF series (SF 96, SF 18) from the company General Electric.

Mention may also be made of polydimethylsiloxanes containing α,ω-omega silanol groups (Dimethiconol according to the CTFA name) such as the oils of the 48 series from the company Rhodia.

In this category of polyalkylsiloxanes, mention may also be made of the products sold under the names Abil Wax 9800 and 9801 by the company Goldschmidt, which are poly(C₁-C₂₀)alkylsiloxanes.

The silicone gums that may be present in the composition according to the invention are notably polydiorganosiloxanes with high number-average molecular masses of between 200 000 and 1 000 000, used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane gums,
polydimethylsiloxane/methylvinylsiloxane gums,
polydimethylsiloxane/diphenylsiloxane gums,
polydimethylsiloxane/phenylmethylsiloxane gums,
polydimethylsiloxane/diphenyl siloxane/methylvinylsiloxane gums.

Products that may be used more particularly are the following mixtures:
mixtures formed from a polydimethylsiloxane hydroxylated at the end of the chain (known as dimethiconol according to the nomenclature of the CTFA dictionary) and from a cyclic polydimethylsiloxane (known as cyclomethicone according to the nomenclature of the CTFA dictionary), such as the product Q2 1401 sold by the company Dow Corning,
mixtures formed from a polydimethylsiloxane gum with a cyclic silicone, such as the product SF 1214 Silicone Fluid from the company General Electric, this product being an SF 30 gum corresponding to a dimethicone, having a number-average molecular weight of 500 000, dissolved in the oil SF 1202 Silicone Fluid corresponding to decamethylcyclopentasiloxane,
mixtures of two PDMSs with different viscosities, and more particularly of a PDMS gum and a PDMS oil, such as the product SF 1236 from the company General Electric. The product SF 1236 is a mixture of a gum SE 30 defined above, with a viscosity of 20 m²/s and of an oil SF 96 with a viscosity of 5×10⁻⁶ m²/s. This product preferably includes 15% of gum SE 30 and 85% of an oil SF 96.

The organomodified non-amino silicones optionally present in the composition according to the invention are non-amino silicones as defined previously and including in their structure one or more organofunctional groups attached via a hydrocarbon-based group.

Among the organomodified non-amino silicones, mention may be made of polyorganosiloxanes including:
polyethyleneoxy and/or polypropyleneoxy groups optionally including C₆ to C₂₄ alkyl groups, such as the products known as dimethicone copolyol sold by the company Dow Corning under the name DC 1248 or the oils Silwet L 722, L 7500, L 77 and L 711 from the company Union Carbide, and the (C₁₂)alkylmethicone copolyol sold by the company Dow Corning under the name Q2 5200,
thiol groups, such as the products sold under the names GP 72 A and GP 71 from the company Genesee,
alkoxylated groups, such as the product sold under the name Silicone Copolymer F-755 by SWS Silicones and Abil Wax 2428, 2434 and 2440 by the company Goldschmidt,
hydroxylated groups, such as the polyorganosiloxanes containing a hydroxyalkyl function, described in French patent application FR 2 589 476,
acyloxyalkyl groups, for instance the polyorganosiloxanes described in patent U.S. Pat. No. 4,957,732,
anionic groups of the carboxylic type, for instance in the products described in patent EP 186 507 from the company Chisso Corporation, or of the alkylcarboxylic type, such as those present in the product X-22-3701E from the company Shin-Etsu; 2-hydroxyalkyl sulfonate; 2-hydroxyalkyl thiosulfate such as the products sold by the company Goldschmidt under the names Abil S201 and Abil S255,
hydroxyacylamino groups, for instance the polyorganosiloxanes described in patent application EP 342 834. Mention may be made, for example, of the product Q2-8413 from the company Dow Corning.

Preferably, the additional silicones are chosen from polydimethylsiloxanes (INCI name: dimethicone), in particular bearing a trimethyloxy end group.

When the non-amino, non-phenyl silicones are non-volatile, they may also be chosen from silicone resins.

Examples that may be mentioned include:
siloxysilicates, which may be trimethyl siloxysilicates of formula [(CH₃)₃XSiXO]ₓX(SiO_{4/2})_{y} (MQ units) in which x and y are integers ranging from 50 to 80,
polysilsesquioxanes of formula (CH₃SiO_{3/2})ₓ (T units) in which x is greater than 100 and at least one of the methyl radicals of which may be substituted with a group R as defined above,
polymethylsilsesquioxanes, which are polysilsesquioxanes in which none of the methyl radicals is substituted with another group. Such polymethylsilsesquioxanes are described in U.S. Pat. No. 5,246,694, the content of which is incorporated by reference.

As examples of commercially available polymethylsilsesquioxane resins, mention may be made of those sold:
by the company Wacker under the reference Resin MK, such as Belsil PMS MK: polymer comprising CH₃SiO_{3/2} repeating units (T units), which may also comprise up to 1% by weight of (CH₃)₂SiO_{2/2} units (D units) and having an average molecular weight of about 10 000,
by the company Shin-Etsu under the references KR-220L, which are composed of T units of formula CH₃SiO_{3/2} and contain Si—OH (silanol) end groups, under the reference KR-242A, which comprise 98% of T units and 2% of dimethyl D units and contain Si—OH end groups, or else under the reference KR-251, comprising 88% of T units and 12% of dimethyl D units and contain Si—OH end groups,
by the company Dow Corning under the reference Dow Corning 680 ID Fluid.

Siloxysilicate resins that may be mentioned include trimethyl siloxysilicate (TMS) resins optionally in the form of powders. Such resins are sold under the reference SR1000 by the company General Electric or under the reference TMS 803 by the company Wacker. Mention may also be made of trimethyl siloxysilicate resins sold in a solvent such as cyclomethicone, sold under the name KF-7312J by the company Shin-Etsu or DC 749 and DC 593 by the company Dow Corning.

The composition according to the invention may also comprise one or more additional compounds.

As additional compounds, which are notably usually used in the cosmetic field, and which may thus be used in accordance with the invention, mention may be made of cationic, anionic, nonionic or amphoteric polymers and mixtures thereof, cationic, anionic, nonionic, amphoteric and/or zwitterionic surfactants and mixtures thereof, antidandruff agents, anti-seborrhoeic agents, agents for preventing hair loss and/or for promoting hair regrowth, fatty substances other than the hydrocarbon-based oils and the silicones defined above, vitamins and provitamins including panthenol, sunscreens, sequestrants, plasticizers, solubilizers, acidifying agents, antioxidants, oxy acids, fragrances, preserving agents and ceramides.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

The Cosmetic Treatment of Keratin Fibres

A subject of the present invention is also a cosmetic process for treating keratin fibres, in particular human keratin fibres such as the hair, comprising the application to said keratin fibres of a cosmetic composition as defined previously.

The composition may be applied to dry or wet keratin fibres that have optionally been washed with shampoo. Preferably, the composition according to the invention is applied to wet keratin fibres.

On conclusion of the cosmetic treatment, the keratin fibres are optionally rinsed with water, optionally washed with a shampoo and then rinsed with water, before being dried or left to dry.

The composition according to the present invention is applied with a leave-on time that may range from 1 to 15 minutes, preferably from 2 to 10 minutes.

When the composition is in the form of a double phase (i.e. it contains two phases that are distinct from each other), it is stirred mechanically so as to perform extemporaneous mixing of the two phases immediately before application.

Use

The present invention also relates to the use of a cosmetic composition as defined previously for conditioning keratin fibres, in particular human keratin fibres such as the hair.

The examples that follow serve to illustrate the invention without, however, being limiting in nature.

### EXAMPLES

In the examples that follow, all the amounts are given, unless otherwise indicated, as mass percentages of active material relative to the total weight of the composition.

### Example 1

a) Test Compositions

Compositions (A1) and (A2) according to the invention, and the comparative compositions (B1) and (B2) below were prepared from the ingredients of which the contents are shown in the table below as mass percentages of starting material relative to the total weight of the composition.

TABLE 1

| | A1 | A2 | B1 | B2 |
|---|---|---|---|---|
| | invention | invention | comparative | comparative |
| Hydrogenated | 7 | 5 | — | — |
| styrene/methyl- | | | | |
| styrene/indene | | | | |
| copolymer ⁽¹⁾ | | | | |
| Bis-cetearyl | 0.5 | 0.5 | — | 0.5 |
| amodimethicone ⁽²⁾ | | | | |
| Trimethylpenta- | 1.75 | 1.75 | 1.75 | 1.75 |
| phenyltrisiloxane ⁽³⁾ | | | | |
| Isododecane | qs 100 | qs 100 | qs 100 | qs 100 |

⁽¹⁾ Regalite R1100 CG hydrocarbon resin - Eastman Chemical
⁽²⁾ Silsoft AX - Momentive
⁽³⁾ Dow Corning PH-1555 HRI Cosmetic Fluid - Dow Corning

b) Procedure

Locks of natural chestnut-brown hair underwent a preliminary relaxing treatment, to simulate degradation of the hair without changing the colour, before being washed with shampoo and then rinsed.

On conclusion of the rinsing, compositions (A1), (A2), (B1) and (B2) above were applied to the locks of “degraded” wet hair in a proportion of 0.15 g of composition per gram of lock.

The locks thus treated were directly dried with a hairdryer without intermediate rinsing.

The sheen of each of the locks, and also that of locks of natural hair and of “degraded” hair, i.e. hair which has undergone a relaxing treatment, but not treated according to the invention were measured using Samba equipment (Bossa Nova Technologies) composed of a polarized light source, a polarized camera and a cylindrical lock holder. Software translates the parameters measured by the system into arbitrary gloss units (BNT).

c) Results

The results of the gloss measurements obtained for each of the locks treated with compositions (A1) and (A2) according to the invention and the comparative compositions (B1) and (B2), and also those of natural and degraded locks, are given in FIG. 1.

The results obtained show that the compositions according to the present invention (A1) and (A2), comprising a combination of a hydrocarbon-based resin and of an aminosilicone, give the hair greater sheen than the comparative compositions (B1) and (B2) not comprising this particular combination.

Compositions (A1) and (A2) significantly improve the sheen of the degraded hair. Composition (A1) even makes it possible to restore sheen of a level similar to that of the natural hair, or even to improve it.

### Example 2

a) Test Compositions

the composition (A3) according to the invention, and the comparative composition (B3) below were prepared from the ingredients of which the contents are shown in the table below as mass percentages of active material relative to the total weight of the composition.

TABLE 2

| | A3 | B3 |
|---|---|---|
| | invention | comparative |
| Isononyl isononanoate | 1.3 | 1.3 |
| Hydrogenated polyisobutene | 8.045 | 9.4 |
| Dimethicone (and) Dimethicone | 6.4 | 6.4 |
| crosspolymer | | |
| Poly dimethylsiloxane 500 000 CST | 11.8 | 11.8 |
| Amodimethicone | 0.4 | 0.4 |
| Hydrogenated Styrene/methyl | 1.355 | — |
| styrene/indene copolymer | | |
| Isododecane | qs 100 | qs 100 |

b) Procedure

Each of the compositions (A3) and (B3) above were applied to locks of sensitized wet brown hair (HT4) in a proportion of 0.4 g of composition per gram of lock. The locks thus treated were then rinsed with water, and dried with a hairdryer by brushing.

The sheen of each of the locks, and also that of locks of natural hair a, i.e. hair which has been sensitized, but not treated according to the invention were measured using Samba equipment (Bossa Nova Technologies) composed of a polarized light source, a polarized camera and a cylindrical lock holder. Software translates the parameters measured by the system into arbitrary gloss units (BNT).

c) Results

The gloss measurements obtained for each of the locks before and after treatment are expressed, arbitrary gloss units (BNT), in table 3 here below.

TABLE 3

| | A3 | B3 |
|---|---|---|
| | invention | comparative |
| Natural sensitized hair (i.e. | 16.57 ± 0.99 | 19.51 ± 1.10 |
| before treatment) | | |
| After treatment | 70.59 ± 17.50 | 41.01 ± 10.68 |
| Improvement before/after | +54.02 | +21.05 |
| treatment | | |

The results thus obtained show that the claimed composition (A3) confers more sheen to the hair than the comparative composition (B3).

1. A cosmetic composition comprising:
(a) one or more hydrophobic film-forming polymers chosen from indene hydrocarbon-based resins, aliphatic pentanediene resins, mixed resins of pentanediene and of indene, diene resins of cyclopentanediene dimers, diene resins of isoprene dimers and mixtures thereof,
(b) one or more aminosilicones, and
(c) one or more organic solvents.

2. The composition according to claim 1, wherein the hydrophobic film-forming polymer(s) (a) are chosen from indene hydrocarbon-based resins, aliphatic pentanediene resins, and mixtures thereof.

3. The composition according to claim 1 wherein the total content of said hydrophobic film-forming polymer(s) (a) ranges from 0.1% to 20% by weight relative to the total weight of the composition.

4. The composition according to claim 1 wherein the aminosilicone(s) (b) are chosen from:
a) the polysiloxanes corresponding to formula (I): 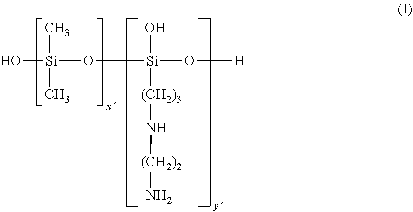
in which x′ and y′ are integers such that the weight-average molecular mass (Mw) is between 5000 and 500 000 g/mol;
b) the aminosilicones corresponding to formula (II):
R′ₐG₃₋ₐ-Si(OSiG₂)ₙ-(OSiG_{b}R′_{2-b})ₘ—O-SiG_{3-a′}-R′_{a′} (II)
in which:
G, which may be identical or different, denotes a hydrogen atom or a phenyl, OH, C₁-C₈ alkyl or C₁-C₈ alkoxy,
a and a′, which may be identical or different, denote 0 or an integer from 1 to 3, in particular 0, with the proviso that at least one from among a and a′ is equal to zero,
b denotes 0 or 1,
m and n are numbers such that the sum (n+m) ranges from 1 to 2000, n denoting a number from 0 to 1999, and m denoting a number from 1 to 2000;
R′, which may be identical or different, denotes a monovalent radical of formula —C_{q}H_{2q}L in which q is a number ranging from 2 to 8 and L is an optionally quaternized amino group chosen from the following groups:
—NR″-Q-N(R″)₂,
—N(R″)₂,
—N⁺(R″)₃A⁻,
—N⁺H(R″)₂A⁻,
—N⁺H₂(R″)A⁻,
—NR″-Q-N⁺(R″)H₂ A⁻,
—NR″-Q-N⁺(R″)₂H A⁻ and
—NR″-Q-N⁺(R″)₃ A⁻,
in which R″, which may be identical or different, denotes hydrogen, phenyl, benzyl, or a saturated monovalent hydrocarbon-based radical; Q denotes a linear or branched group of formula CᵣH₂ᵣ, r being an integer ranging from 2 to 6; and A⁻ represents a cosmetically acceptable anion;
c) the aminosilicones corresponding to formula (VIII): 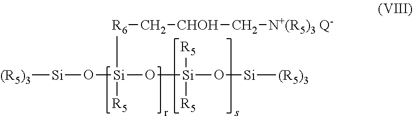
in which:
R₅ represents a monovalent hydrocarbon-based radical containing from 1 to 18 carbon atoms, or C₂-C₁₈ alkenyl;
R₆ represents a divalent hydrocarbon radical;
Q⁻ is an anion such as a halide ion or an organic acid salt;
r represents a mean statistical value ranging from 2 to 20;
s represents a mean statistical value ranging from 20 to 200;
d) the quaternary ammonium silicones of formula (IX): 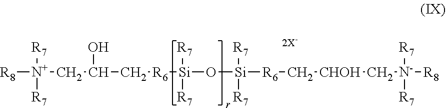
in which:
R₇, which may be identical or different, represent a monovalent hydrocarbon-based radical containing from 1 to 18 carbon atoms, a C₂-C₁₈ alkenyl radical or a ring comprising 5 or 6 carbon atoms;
R₆ represents a divalent hydrocarbon radical;
R₈, which may be identical or different, represent a hydrogen atom, a monovalent hydrocarbon-based radical containing from 1 to 18 carbon atoms, a C₂-C₁₈ alkenyl substituent or a —R₆—NHCOR₇ radical;
X⁻ is an anion such as a halide ion or an organic acid salt;
r represents a mean statistical value ranging from 2 to 200;
e) the aminosilicones of formula (X): 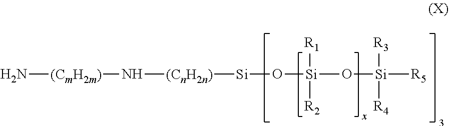
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;
f) multiblock polyoxyalkylenated aminosilicones, of the type (AB)ₙ, A being a polysiloxane block and B being a polyoxyalkylenated block including at least one amine group,
g) the aminosilicones of formulae (XI) and (XII): 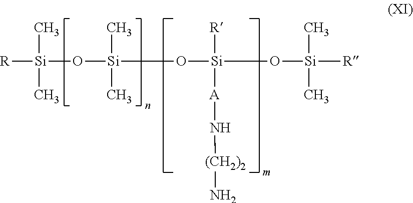
in which:
R, R′ and R″, which may be identical or different, denote a C₁-C₄ alkyl group or a hydroxyl group,
A denotes a C₃ alkylene radical; and
m and n are numbers such that the weight-average molecular mass of the compound is between 5000 and 500 000 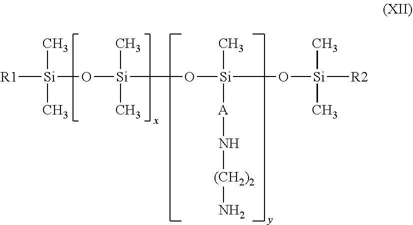
in which:
x and y are numbers ranging from 1 to 5000;
R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.
h) polysiloxanes and notably polydimethylsiloxanes including primary amine groups at only one chain end or on side chains,
i) and mixtures thereof.

5. The composition according to claim 1 wherein the aminosilicone(s) (b) are chosen from the aminosilicones of formulae (II) and (XII) and the polysiloxanes of formula (XVI):

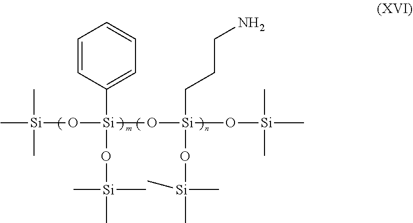

6. The composition according to claim wherein the aminosilicone(s) (b) are chosen from the aminosilicones of formula (XII), and mixtures thereof.

7. The composition according to claim 1, wherein the content of the aminosilicone(s) (b) ranges from 0.05% to 10% by weight relative to the total weight of the composition.

8. The composition according to claim 1 wherein the weight ratio (Ra) between the total content of said hydrophobic film-forming polymer(s) (a) and the total content of the aminosilicone(s) (b) is greater than or equal to 1.

9. The composition according to claim 1 wherein the organic solvent(s) are chosen from hydrocarbon-based oils and mixtures thereof.

10. The composition according to claim 1 wherein the content of the organic solvent(s) ranges from 10% to 99% by weight relative to the total weight of the composition.

11. The composition according to claim 1 wherein it also comprises one or more silicones other than the aminosilicone(s) (b) and comprising one or more phenyl groups.

12. The composition according to claim 11, wherein the content of the silicone(s) other than the aminosilicone(s) (b) and comprising one or more phenyl groups ranges from 0.1% to 10% by weight, relative to the total weight of the composition.

13. The composition according to claim 11, wherein the weight ratio (Rb) between the total content of said hydrophobic film-forming polymer(s) (a) and the total content of the silicone(s) other than the aminosilicone(s) (b) and comprising one or more phenyl groups is greater than or equal to 1.

14. The composition according to claim 1 which is anhydrous.

15. The composition according to claim 1 which also comprises one or more dyestuffs.

16. A cosmetic process for treating keratin fibres comprising the application to said keratin fibres of a cosmetic composition as defined in claim 1.

17. A cosmetic process for conditioning keratin fibres comprising the application to said keratin fibres of a cosmetic composition as defined in claim 1.

18. The composition according to claim 2, wherein the total content of said hydrophobic film-forming polymer(s) (a) ranges from 0.1% to 20% by weight to the total weight of the composition.

19. The composition according to claim 2, wherein the aminosilicone(s) (b) are chosen from:
a) the polysiloxanes corresponding to formula (I): 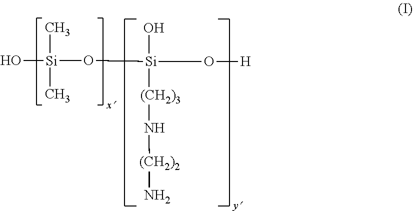
in which x′ and y′ are integers such that the weight-average molecular mass (Mw) is between 5000 and 500 000 g/mol;
b) the aminosilicones corresponding to formula (II):
R′ₐG₃₋ₐ-Si(OSiG₂)ₙ-(OSiG_{b}R′_{2-b})ₘ—O-SiG_{3-a′}-R′_{a′} (II)
in which:
G, which may be identical or different, denotes a hydrogen atom or a phenyl, OH, C₁-C₈ alkyl or C₁-C₈ alkoxy,
a and a′, which may be identical or different, denote 0 or an integer from 1 to 3, in particular 0, with the proviso that at least one from among a and a′ is equal to zero,
b denotes 0 or 1,
m and n are numbers such that the sum (n+m) ranges from 1 to 2000 n denoting a number from 0 to 1999, and m denoting a number from 1 to 2000;
R′, which may be identical or different, denotes a monovalent radical of formula —C_{q}H_{2q}L in which q is a number ranging from 2 to 8 and L is an optionally quaternized amino group chosen from the following groups:
—NR″-Q-N(R″)₂,
—N(R″)₂,
—N⁺(R″)₃ A⁻,
—N⁺H(R″)₂A⁻,
—N⁺H₂(R″)A⁻,
—NR″-Q-N⁺(R″)H₂A⁻,
—NR″-Q-N⁺(R″)₂H A⁻ and
—NR″-Q-N⁺(R″)₃ A⁻,
in which R″, which may be identical or different, denotes hydrogen, phenyl, benzyl, or a saturated monovalent hydrocarbon-based radical; Q denotes a linear or branched group of formula CᵣH₂ᵣ, r being an integer ranging from 2 to 6; and A″ represents a cosmetically acceptable anion;
c) the aminosilicones corresponding to formula (VIII): 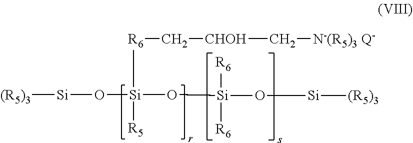
in which:
R₅ represents a monovalent hydrocarbon-based radical containing from 1 to 18 carbon atoms, or C₂-C₁₈ alkenyl;
R₆ represents a divalent hydrocarbon radical;
Q⁻ is an anion such as a halide ion or an organic acid salt;
r represents a mean statistical value ranging from 2 to 20;
s represents a mean statistical value ranging from 20 to 200;
d) the quaternary ammonium silicones of formula (IX): 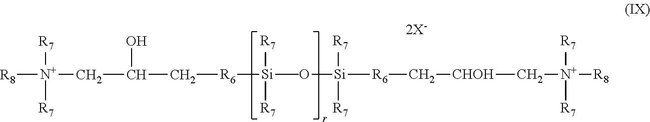
in which:
R₇, which may be identical or different, represent a monovalent hydrocarbon-based radical containing from 1 to 18 carbon atoms, a C₂-C₁₈ alkenyl radical or a ring comprising 5 or 6 carbon atoms;
R₆ represents a divalent hydrocarbon radical;
R₈, which may be identical or different, represent a hydrogen atom, a monovalent hydrocarbon-based radical containing from 1 to 18 carbon atoms, a C₂-C₁₈ alkenyl substituent or a —R₆—NHCOR₇ radical;
X⁻ is an anion such as a halide ion or an organic acid salt;
r represents a mean statistical value ranging from 2 to 200;
e) the aminosilicones of formula (X): 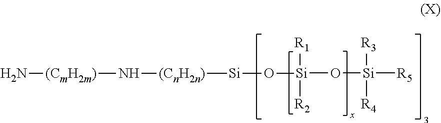
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;
f) multiblock polyoxyalkylenated aminosilicones, of the type (AB)ₙ, A being a polysiloxane block and B being a polyoxyalkylenated block including at least one amine group,
g) the aminosilicones of formulae (XI) and (XII): 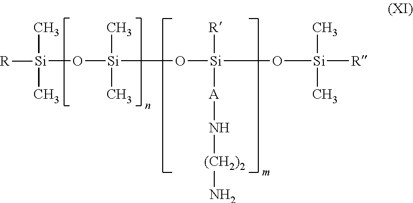
in which:
R, R′ and R″, which may be identical or different, denote a C₁-C₄ alkyl group or a hydroxyl group,
A denotes a C₃ alkylene radical; and
m and n are numbers such that the weight-average molecular mass of the compound is between 5000 and 500 000 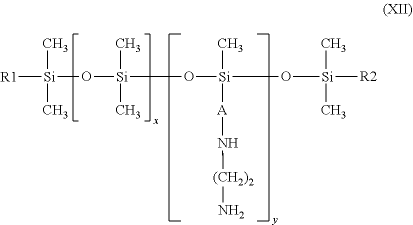
in which:
x and y are numbers ranging from 1 to 5000;
R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.
h) polysiloxanes and notably polydimethylsiloxanes including primary amine groups at only one chain end or on side chains,
i) and mixtures thereof.

20. The composition according to claim 3 wherein the aminosilicone(s) (b) are chosen from:
a) the polysiloxanes corresponding to formula (I): 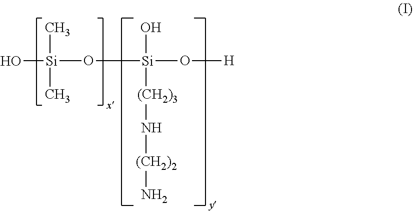
in which x′ and y′ are integers such that the weight-average molecular mass (Mw) is between 5000 and 500 000 g/mol;
b) the aminosilicones corresponding to formula (II):
R′ₐG₃₋ₐ-Si(OSiG₂)ₙ-(OSiG_{b}R′_{2-b})ₘ—O-SiG_{3-a′}-R′_{a′} (II)
in which:
G, which may be identical or different, denotes a hydrogen atom or a phenyl, OH, C₁-C₈ alkyl or C₁-C₈ alkoxy,
a and a′, which may be identical or different, denote 0 or an integer from 1 to 3, in particular 0, with the proviso that at least one from among a and a′ is equal to zero,
b denotes 0 or 1,
m and n are numbers such that the sum (n+m) ranges from 1 to 2000 n denoting a number from 0 to 1999, and m denoting a number from 1 to 2000;
R′, which may be identical or different, denotes a monovalent radical of formula —C_{q}H_{2q}L in which q is a number ranging from 2 to 8 and L is an optionally quaternized amino group chosen from the following groups:
—NR″-Q-N(R″)₂,
—N(R″)₂,
—N⁺(R″)₃ A⁻,
—N⁺H(R″)₂ A⁻,
—N⁺H₂(R″) A⁻,
—NR″-Q-N⁺(R″)H₂ A⁻,
—NR″-Q-N⁺(R″)₂H A⁻ and
—NR″-Q-N⁺(R″)₃ A⁻,
in which R″, which may be identical or different, denotes hydrogen, phenyl, benzyl, or a saturated monovalent hydrocarbon-based radical; Q denotes a linear or branched group of formula CᵣH₂ᵣ, r being an integer ranging from 2 to 6; and A⁻ represents a cosmetically acceptable anion;
c) the aminosilicones corresponding to formula (VIII): 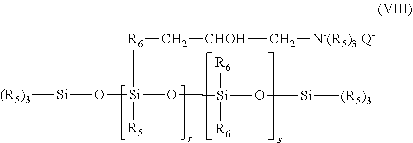
in which:
R₅ represents a monovalent hydrocarbon-based radical containing from 1 to 18 carbon atoms, or C₂-C₁₈ alkenyl;
R₆ represents a divalent hydrocarbon radical;
Q⁻ is an anion such as a halide ion or an organic acid salt;
r represents a mean statistical value ranging from 2 to 20;
s represents a mean statistical value ranging from 20 to 200;
d) the quaternary ammonium silicones of formula (IX): 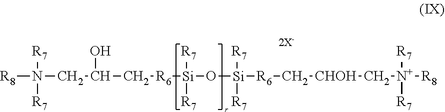
in which:
R₇, which may be identical or different, represent a monovalent hydrocarbon-based radical containing from 1 to 18 carbon atoms, a C₂-C₁₈ alkenyl radical or a ring comprising 5 or 6 carbon atoms;
R₆ represents a divalent hydrocarbon radical;
R₈, which may be identical or different, represent a hydrogen atom, a monovalent hydrocarbon-based radical containing from 1 to 18 carbon atoms, a C₂-C₁₈ alkenyl substituent or a —R₆—NHCOR₇ radical;
X⁻ is an anion such as a halide ion or an organic acid salt;
r represents a mean statistical value ranging from 2 to 200;
e) the aminosilicones of formula (X): 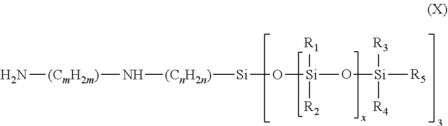
in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g;
f) multiblock polyoxyalkylenated aminosilicones, of the type (AB)ₙ, A being a polysiloxane block and B being a polyoxyalkylenated block including at least one amine group,
g) the aminosilicones of formulae (XI) and (XII): 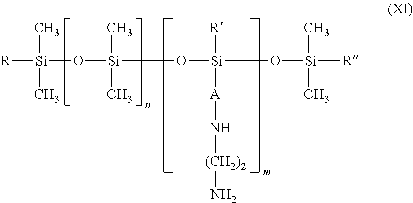
in which:
R, R′ and R″, which may be identical or different, denote a C₁-C₄ alkyl group or a hydroxyl group,
A denotes a C₃ alkylene radical; and
m and n are numbers such that the weight-average molecular mass of the compound is between 5000 and 500 000 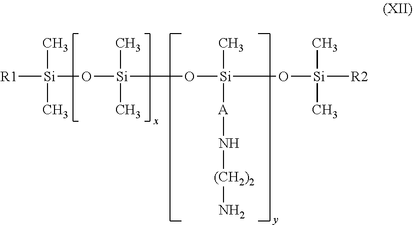
in which:
x and y are numbers ranging from 1 to 5000;
R₁ and R₂, which may be identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical containing from 2 to 8 carbon atoms.
h) polysiloxanes and notably polydimethylsiloxanes including primary amine groups at only one chain end or on side chains,
i) and mixtures thereof.
